(19) United States
(12) Patent Application Publication
(10) Pub. No.: US 2025/0332342 A1
(43) Pub. Date: Oct. 30, 2025

(54) Implantable Infusion Pump

(71) Applicant: Henry Buchwald, Edina, MN (US)

(72) Inventor: Henry Buchwald, Edina, MN (US)

(21) Appl. No.: 19/188,338

(22) Filed: Apr. 24, 2025

Related U.S. Application Data
(60) Provisional application No. 63/637,947, filed on Apr. 24, 2024.

(51) Int. Cl. A61M 5/155; A61M 5/142

(52) U.S. Cl. CPC A61M 5/155; A61M 5/14276; A61M 2202/0007; A61M 2205/04; A61M 2205/8225

(57) ABSTRACT

The invention is an improved implantable infusion pump of the type having a sealed outer/inferior chamber holding a fluorocarbon, wherein the improvement(s) are replacing the fluorocarbon with a fluorocarbon mixture with an uniform rate of expansion (+/−3%) over a temperature range of from 85° F. to 105° F., or replacing the fluorocarbon with a different chemical exhibiting comparable vapor/liquid and expansion capability with a uniform rate of expansion (+/−3%), over a temperature range of from 85° F. to 105° F. One example of such a mixture for the first improvement is a binary n-decafluorobutane (Halocarbon 610 or refrigerant R 610) and n-perfluorohexane (Fluorinert FC-72) with a weight/weight ratio falling between 25/75 and 35/65. Three such examples of the second improvement are 2-methylbutane, 1-pentane, and a 50:50 mixture of the two.

### CROSS-REFERENCE TO RELATED APPLICATIONS

This application is an improvement to U.S. Pat. No. 3,731,681 which issued May 8, 1973, and provisional application No. 63/461,741, filed Apr. 25, 2023, and claims priority to provisional application 63/637,947, filed Apr. 24, 2024, the entire contents of all of which are hereby incorporated by reference.

### FIELD OF THE INVENTION

The invention is an improved implantable infusion pump utilizing a fluorocarbon mixture to extend the temperature range over which the devices operates therapeutically. The invention encompasses two improvements to the implantable infusion pump utilizing a fluorocarbon mixture to extend the temperature range over which the device operates therapeutically, or replacing the fluorocarbon with another or a mixture of complimentary vapor/liquid chemical power sources that are less toxic to the environment, particularly the ozone layer

### BACKGROUND OF THE INVENTION

Applicant was one of the inventors on U.S. Pat. No. 3,731,681, which was the first implantable infusion pump and is the only FDA approved pump for hepatic arterial chemotherapy.

The pump disclosed in U.S. Pat. No. 3,731,681 was a two-chambered device having a sealed outer/inferior chamber holding the propellant, the chemical power source, a fluorocarbon, that expands at a fixed rate from a liquid to a vapor governed by pressure and temperature. At one atmosphere, our normal state, it is only temperature dependent. As the propellant expands to vapor it exerts ever-increasing pressure on a bellows mechanism that transfers that pressure to the inner/superior chamber that contains the infusion chemical (chemotherapy agent, analgesic, insulin, etc.), which is not sealed, but, as the pressure of the bellows increases, expels the infusion substance via a catheter into the patient's artery, vein, cerebral spinal space, or body cavity. When nearly empty, the infusion chamber is filled with a new infusion substance via a self-sealing port and special needle (Huber) from a syringe, the kinetic pressure on the syringe plunger forcing the fluid into the inner/superior chamber, which, in turn, via the action of the reverse bellows mechanism, restricts the sealed outer/inferior chamber volume, thereby, reconstituting the fluorocarbon in the sealed chamber back into the smaller volume liquid phase from the vapor phase. The advantage of the perpetual power source is to make implantation permanent and free of battery replacement.

A problem with the pump is that the function of the device can become non-therapeutic, with possible toxic or inadequate flow rates at temperatures outside normal patient ranges. If the patient has a fever, the flow rate might be too fast, forcing physicians to advise the remedy of an icepack over the pump site, even immediate hospitalization. If the patient is exposed to severe cold, the flow rate might be too slow, forcing physicians to advise hotpacks over the pump site or a hot bath. A nother problem with the pump is that the fluorocarbon chemical power source has certain toxic properties, among which is when released into the atmosphere can be detrimental to the ozone layer. This property has caused usage precautions and limitations in the United States, as well as the total or partial banning of the use of fluorocarbons in Europe. This stipulation effectively bans the current fluorocarbon pump design from clinical application in Europe.

### SUMMARY OF THE INVENTION

The first improvement to U.S. Pat. No. 3,731,681 is to substitute for the single fluorocarbon in the propellant pump chamber by a fluorocarbon mixture with an uniform rate of expansion (+/−3%). This would keep the flow rate with an acceptable of varying body temperatures, taking into account fevers or chilling.

One example of a fluorocarbon mixture is a binary n-decafluorobutane (Halocarbon 610 or refrigerant R 610) and n-perfluorohexane (Fluorinert FC-72) with a weight/weight ratio falling between 25/75 and 35/65.

Other mixtures of fluorocarbons are also considered part of the invention, in various percentages, with the important factor being a uniform rate of expansion over a proper temperature range, typically from 85° F. to 105° F.

The second improvement to U.S. Pat. No. 3,731,681 is to substitute for the fluorocarbon chemical power source (perfluoropentane) with a non-fluorocarbon chemical source, or a mixture thereof, possessing the desired liquid/vapor expansion/contraction properties (+/−3%) as fluorocarbon in order to keep the flow rate within an acceptable range at varying body temperature range of from 85° F. to 105° F.

One example of a perfluoropentane substitute at body temperatures is 2-methyl butane with a vapor pressure 5% above perfluoropentane.

Another example of a perfluoropentane substitute at body temperatures is 1-pentene with a vapor pressure 5% below perfluoropentane.

A 50:50 mixture of 1-pentene/2-methylbutane would almost exactly match the vapor pressure of perfluoropentane.

### DETAILED DESCRIPTION OF THE INVENTION

While these invention(s) may be embodied in many forms, they are detailed herein specific embodiments of the inventions. These descriptions are an exemplification of the principles of the inventions and are not intended to limit the inventions to the particular embodiments illustrated.

The first invention is an improved implantable infusion pump of the type having a sealed outer/inferior chamber holding a fluorocarbon, wherein the improvement is replacing the fluorocarbon with a fluorocarbon mixture with an uniform rate of expansion (+/−3%) over a temperature range of from 85° F.-105° F. An example of such a mixture is a binary n-decafluorobutane (Halocarbon 610 or refrigerant R 610) and n-perfluorohexane (Fluorinert FC-72) with a weight/weight ratio falling between 25/75 and 35/65.

Other mixtures of fluorocarbons are also considered part of the invention, in various percentages, with the important factor being uniform rate of expansion over a proper temperature range, typically from 85° F. to 105° F.

The second invention is an improved infusion pump of the type having a sealed outer/inferior chamber holding a fluorocarbon substitute with an uniform rate of expansion (+/−3%) over a temperature range of from 85° F. to 105° F. Examples of such substances are: tetramethyl silane, diethyl ether, methyl formate, ethyl amine, trichlorofluoromethane, and nitrogen tetroxide.

Specific examples of such fluorocarbon substitutes in the appropriate vapor pressure are 2-methyl butane, 1-pentane, and a 50:50 mixture of the two.

Other substitute fluorocarbons singly or in mixture are also considered part of the invention, in various percentages, with the important factor being uniform rate of expansion over a proper temperature range typically from 85° F. to 105° F.

What is claimed is:

1. An improved implantable infusion pump of the type having a sealed outer/inferior chamber holding a fluorocarbon or a fluorocarbon substitute, the improvement comprising:
replacing the fluorocarbon with a fluorocarbon mixture with a uniform rate of expansion (+/−3%), over a temperature range of from 85° F. to 105° F.

2. The improved implantable infusion pump of claim 1 wherein the fluorocarbon mixture is a binary n-decafluorobutane (Halocarbon 610 or refrigerant R 610) and n-perfluorohexane (Fluorinert FC-72) with a weight/weight ratio falling between 25/75 and 35/65.

3. The improved implantable infusion pump of claim 1 wherein the fluorocarbon substitute is 2-methylbutane or 1-pentane or a 50:50 mixture thereof.

4. An improved implantable infusion pump of the type having a sealed outer/inferior chamber holding a fluorocarbon or a fluorocarbon substitute, the improvement comprising:
replacing the fluorocarbon with a non-fluorocarbon with comparable intrinsic expansion capability to achieve an uniform rate of expansion (+/−3%), over a temperature range of from 85° F. to 105° F.

5. The improved implantable infusion pump of claim 4 wherein the fluorocarbon mixture is a binary n-decafluorobutane (Halocarbon 610 or refrigerant R 610) and n-perfluorohexane (Fluorinert FC-72) with a weight/weight ratio falling between 25/75 and 35/65.

6. The improved implantable infusion pump of claim 4 wherein the fluorocarbon substitute is 2-methylbutane or 1-pentane or a 50:50 mixture thereof.
